(19) United States
(12) Patent Application Publication
(10) Pub. No.: US 2025/0259301 A1
(43) Pub. Date: Aug. 14, 2025

(54) MEASUREMENT DEVICE AND MEASUREMENT METHOD

(71) Applicant: Sharp Kabushiki Kaisha, Sakai City (JP)

(72) Inventors: KYOHKO MATSUDA, Sakai City (JP); Yoshihisa Adachi, Sakai City (JP); Tetsuya Okumura, Sakai City (JP)

(21) Appl. No.: 19/049,340

(22) Filed: Feb. 10, 2025

(30) Foreign Application Priority Data
Feb. 28, 2024 (JP) 2024-027972

(51) Int. Cl. G06T 7/00; G06T 7/246

(52) U.S. Cl. CPC G06T 7/0012; G06T 7/246; G06T 2207/10016; G06T 2207/30104

(57) ABSTRACT

A measurement device includes the following: a time-series signal obtaining unit configured to obtain a biological signal, which is time-series data of a biological signal value calculated from an image captured by imaging a living body, and obtain a determination index for the image; a biological-information calculating unit configured to divide the biological signal into pulse signals at predetermined times based on the cycle of a biological phenomenon; and a pulse determining unit configured to adopt pulse signals including biological signal vales calculated from the image that enables a determination index satisfying a determination condition to be calculated. The biological-information calculating unit calculates biological information by using the pulse signals adopted by the pulse determining unit.

### CROSS-REFERENCE TO RELATED APPLICATION

The present application claims priority from Japanese Application JP2024-027972, filed on Feb. 28, 2024, the content of which is hereby incorporated by reference into this application.

### BACKGROUND

### 1. Field

The present disclosure relates to a measurement device and a measurement method.

### 2. Description of the Related Art

Japanese Unexamined Patent Application Publication No. 2018-019882 discloses a person-image detecting unit that detects an image of a person, a biological-signal detecting unit that detects a biological signal of the person, and a control unit that outputs a detected result from the biological-signal detecting unit. A vital sign detection device disclosed in Japanese Unexamined Patent Application Publication No. 2018-019882 determines that the biological-signal detecting unit is not faulty, and outputs biological information based on a biological signal in response to a determination that a biological value indicated by the biological signal is within a predetermined normal range, and that there is a person who stays still in a region detected by the person-image detecting unit.

### SUMMARY

The cycle of a life activity sometimes reflects the health of a living body. For instance, biological phenomena, such as heartbeat and respiration, have their cycles of repetition of increase and decrease. Some biological signals obtained by measuring the living body reflect the cycle of such a biological phenomenon, and analyzing the signal's periodicity can offer information on the health of the living body. An exemplary signal is one obtained through measurement with a pulse sensor or a pulse oximeter. However, in the case of a disturbance adversely affecting the measurement, a detected biological signal contains noise, and such a biological signal may not appropriately indicate the periodicity based on a biological phenomenon. It is noted that the disturbance herein indicates not a signal in a specific control system, but an unexpected influence from outside a system, or an undesired external interference received the device.

The technique disclosed in Japanese Unexamined Patent Application Publication No. 2018-019882 uses a pulse sensor and an image detecting device, to output pulse information upon detection of an image of a driver, and not to output the pulse information upon no detection of the driver's image. However, a disturbance, such as a driver's body motion, sometimes occurs discontinuously on a second-by-second basis. Since pulse reflects periodic temporal variation in the heart, the pulse's pulsation varies periodically with time in conformance with the heart; typically, pulse is calculated by detecting the heart's periodicity, and thus, detection based on the period of the pulsation is desirable. However, a failure to detect the image during the period results in an interruption of the pulsation calculation during the period, and thus, the pulse is calculated based on incomplete pulsation with off-balance periodicity. As a result, the technique disclosed in Japanese Unexamined Patent Application Publication No. 2018-019882 may fail to accurately calculate the biological information in the case of a disturbance adversely affecting the measurement. As such, it is an object of one aspect of the present disclosure to provide a measurement device and a measurement method that can calculate accurate biological information reflecting the cycle of a biological phenomenon of the living body.

A measurement device according to one aspect of the present disclosure includes the following: an imaging unit configured to image a living body to capture an image as time-series data; a time-series signal obtaining unit configured to obtain a time-series biological signal indicating a temporal variation in a biological signal, and obtain a determination index temporally associated with the biological signal, the biological signal being a value relating to the living body and calculated from the image; a biological-information calculating unit configured to extract a pulse signal by dividing the time-series biological signal at a predetermined time based on a cycle of a biological phenomenon of the living body; and a pulse determining unit configured to determine whether the determination index satisfies a predetermined determination condition, and adopt the pulse signal temporally associated with the determination index satisfying the predetermined determination condition. The biological-information calculating unit calculates biological information relating to the living body by using the pulse signal adopted by the pulse determining unit.

A measurement method according to another aspect of the present disclosure includes the following: imaging a living body to capture an image as time-series data; obtaining a time-series biological signal indicating a temporal variation in a biological signal, and obtaining a determination index temporally associated with the biological signal, the biological signal being a value relating to the living body and calculated from the image; extracting a pulse signal by dividing the time-series biological signal at a predetermined time based on a cycle of a biological phenomenon of the living body; determining whether the determination index satisfies a predetermined determination condition; and calculating biological information relating to the living body by using the pulse signal temporally associated with the determination index satisfying the predetermined determination condition.

### BRIEF DESCRIPTION OF THE DRAWINGS

FIG. 1 illustrates, by way of example, a measurement device being used;

FIG. 2 is a block diagram illustrating one example of the configuration of a measurement device according to a first embodiment;

FIG. 3 illustrates one example of a region of interest;

FIG. 4A is a graph illustrating, by way of example, transition of a determination index that indicates the number of pixels within the region of interest, and a threshold that is a determination threshold;

FIG. 4B is a graph illustrating, by way of example, pulse signals at time points shown in the transition of the determination index illustrated in FIG. 4A;

FIG. 5 is a flowchart showing one example of the operation of the measurement device according to the first embodiment;

FIG. 6 is a block diagram illustrating one example of the configuration of a measurement device according to a second embodiment;

FIG. 7A is a graph illustrating, by way of example, transition of a determination index that indicates the amount of variation in the measurement device, and a threshold that is a determination threshold;

FIG. 7B is a graph illustrating, by way of example, pulse signals at time points shown in the transition of the amount of variation in the measurement device illustrated in FIG. 7A; and

FIG. 8 is a flowchart showing one example of the operation of the measurement device according to the second embodiment.

### DETAILED DESCRIPTION OF THE DISCLOSURE

### First Embodiment

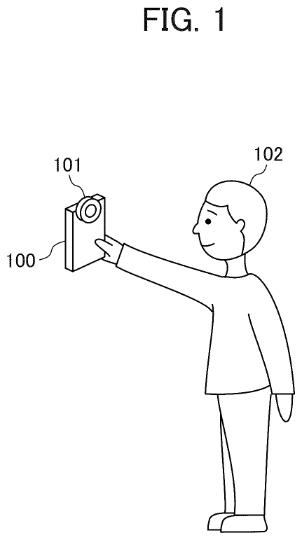

The first embodiment will be described with reference to FIGS. 1 to 5. It is noted that identical or equivalent constituents will be denoted by the same signs throughout the drawings, and the descriptions of redundancies will be omitted.

FIG. 1 illustrates, by way of example, a measurement device 100 being used. As illustrated in FIG. 1, the measurement device 100 includes an imaging unit 101.

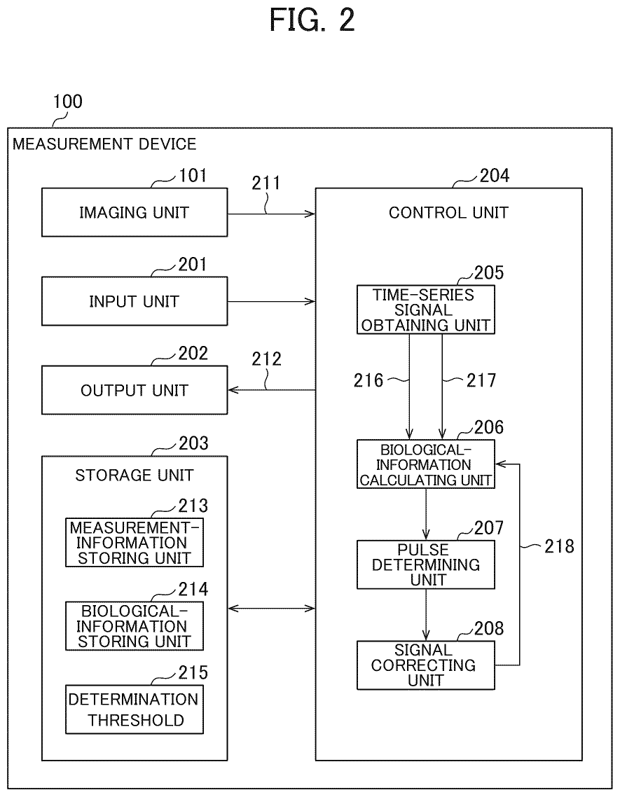

The measurement device 100 uses an image of a living body 102 captured as time-series data by the imaging unit 101, to measure time-series variation in the skin surface or skin inside of the living body 102 to obtain biological information 212 (see FIG. 2). It is noted that the image herein is captured so that a biological signal can be obtained as time-series data; for instance, it is an image of the living body 102 captured in at least a plurality of frames, such as a moving picture, or a still picture obtained as time-series data.

Example of the measurement device 100 include a personal computer (PC), a smartphone, a tablet terminal, a dedicated biological-information measuring terminal, and a watching robot equipped with the imaging unit 101. Example of the biological information 212 include blood pressure, pulse, respiration rate, and blood oxygen saturation. FIG. 1 illustrates the imaging unit 101 imaging the living body 102 with the measurement device 100 held by the living body 102. Nevertheless, the imaging unit 101 may image the living body 102 without the measurement device 100 held by the living body 102.

The imaging unit 101 images the living body 102 to obtain its image as time-series data. The imaging unit 101 is installed in such a location as to be able to image an exposed body surface of the living body 102. Examples of the exposed body surface of the living body 102 include the forehead, cheeks, fingertips, wrists, and palms of the living body 102. The imaging unit 101 is installed in, but not limited to, a PC, a smartphone, a tablet, a display, a mirror, and a washstand.

The imaging unit 101 is a camera including a charge coupled device (CCD), a complementary metal oxide semiconductor (CMOS) image sensor, and a lens. The imaging unit 101 may include a camera image sensor including an RGB filter. For instance, the imaging unit 101 includes an RGB Bayer-arrangement color filter in order to detect a minute change of color of the skin of the living body 102. Alternatively, the imaging unit 101 may include a color filter of RGBCy, RGBIR, or other arrangements. Color filters of RGBCy, RGBIR, and other arrangements are suitable for observing blood volume fluctuation, which is indicated by light transmitted into the skin inside and reflected.

FIG. 2 is a block diagram illustrating one example of the configuration of the measurement device 100 according to this embodiment.

The imaging unit 101 includes, but not limited to, the imaging unit 101, an input unit 201, an output unit 202, a storage unit 203, and a control unit 204. The imaging unit 101, the input unit 201, the output unit 202, and the storage unit 203 are electrically connected to the control unit 204.

The imaging unit 101 images the living body 102 to capture the image 211 and sends the captured image 211 to the control unit 204. For instance, the imaging unit 101 images the living body 102 to capture the image 211 at 30 to 60 frames per second (fps). The image 211 includes an image of the body surface of the living body 102.

The input unit 201 receives an input of information necessary for the measurement device 100. Examples of the input unit 201 include a keyboard, a mouse, and a touch panel.

The output unit 202 outputs, but not limited to, the image 211, the biological information 212, a message to the living body 102, and the date and time of imaging of the image 211. For instance, the output unit 202 includes, but not limited to, a display and a speaker.

The control unit 204 executes various kinds of processing in accordance with a program and data stored in the storage unit 203. The control unit 204 is formed from processors, such as a central processing unit (CPU) and a graphic processing unit (GPU).

The control unit 204 includes a time-series signal obtaining unit 205, a biological-information calculating unit 206, a pulse determining unit 207, and a signal correcting unit 208.

The time-series signal obtaining unit 205 calculates a biological signal from the image 211. Since the image 211 is captured as time-series data, which is temporal variation for each frame of the image of the living body 102, the time-series signal obtaining unit 205 can obtain a time-series biological signal 217, which is time-series data of a biological signal value calculated from the image 211. For instance, the biological signal value is a representative value or average value of the pixel values of a plurality of pixels included in a region of interest ROI, and the time-series biological signal 217 is temporal variation in the biological signal value. In the time-series biological signal 217, a time-series biological signal value, i.e., the biological signal value calculated from the image 211, is associated with the time point of obtainment of the biological signal value. Here, the time point of obtainment of the biological signal value is defined as a time point when the imaging unit 101 captures a frame with the biological signal value calculated in the image 211. The biological signal value is actually obtained through the following: the time-series signal obtaining unit 205 sets the region of interest ROI in a predetermined frame of the image 211 and then calculates the biological signal value from the pixel values of the pixels included in the region of interest ROI. Since the time point of imaging of the living body 102 is the timing of obtainment of the biological signal value reflecting the conditions of the living body 102 at that time, a timing when the imaging unit 101 captures a frame with the biological signal value calculated is herein defined as a time point when the biological signal value is obtained from the frame. Although the biological signal value can be calculated without performing, for each frame, the setting of the region of interest ROI, and the calculation using the image within the region of interest ROI, these processes are desirably performed for each frame because doing so at shorter time intervals can obtain the biological signal as time-series data more accurately. For this reason, the biological signal value in this embodiment is obtained for each frame and obtained as time-series data.

Furthermore, the time-series signal obtaining unit 205 obtains a determination index for the image 211 at predetermined timings to obtain a determination index signal 216, which is time-series data of the determination index. The determination index according to this embodiment indicates the number of pixels within the region of interest ROI included in the image 211. In the present disclosure, the number of pixels is information indicating the number of pixels included in the image 211; for instance, it is the number of subpixels for each of red (R), green (G), and blue (B) in an RGB space, or the number of pixels calculated from a subpixel value.

The biological-information calculating unit 206 obtains pulse signals by temporally dividing the time-series biological signal 217 on the basis of the cycle of a biological phenomenon that a user wants to detect. Each pulse signal is thus temporally associated with the time point of obtainment of the biological signal value included in the pulse signal. Exemplary biological phenomena that can be a detection target include pulse, heartbeat, respiration, blood pressure fluctuation, and blood enzyme, and they are determined in accordance with the type of the biological information 212 that the user wants to detect.

The biological-information calculating unit 206 calculates the biological information 212 on the basis of a correction signal 218, which is generated by the signal correcting unit 208. The biological information 212 indicates information about the living body 102 that the user wants to detect. For instance, the biological information 212 indicates his/her blood pressure, heart rate, or blood oxygen saturation.

The pulse determining unit 207 determines whether the pulse signal satisfies a determination condition on the basis of a determination index value obtained from the determination index signal 216. The pulse determining unit 207 then adopts pulse signals temporally associated with determination indices satisfying the determination condition. The determination condition according to this embodiment is that the number of pixels within the region of interest ROI exceeds a determination threshold 215 (a first determination threshold) according to this embodiment.

The signal correcting unit 208 generates the correction signal 218 formed from the pulse signals adopted by the pulse determining unit 207.

The storage unit 203 is a recording medium that can record, but not limited to, various data pieces and programs and is a hard disk, a solid state driver (SSD), a semiconductor memory, or other things. The storage unit 203 includes a measurement-information storing unit 213, a biological-information storing unit 214, and a determination threshold 215. The storage unit 203 may store a plurality of determination thresholds 215.

The measurement-information storing unit 213 stores, but not limited to, a pre-stored program and a user-registered information piece that are necessary for measuring the biological information 212. For instance, the measurement-information storing unit 213 stores a calculation formula relating to the biological information 212, and a calculation formula relating to the determination index. For instance, the user is the living body 102 or an administrator of the measurement device 100.

The biological-information storing unit 214 stores, as adopted-pulse information, information that enables identifying which of all the pulse signals are adopted pulse signals. For example, an adopted pulse signal itself, a unique number that enables identifying the adopted pulse signal, information, such as the time point of obtainment of the determination index temporally associated with the adopted pulse signal, or a biological signal value included in the adopted pulse signal is stored as the adopted-pulse information. The adopted-pulse information may include other information relating to the determination index. For example, the adopted-pulse information may include a time when the determination index satisfies the determination condition, or a unique number that enables identifying the time when the determination index satisfies the determination condition.

At least one of the measurement-information storing unit 213 and biological-information storing unit 214 may store, but not limited to, information relating to the living body 102. For example, the information relating to the living body 102 indicates a log of the biological information 212.

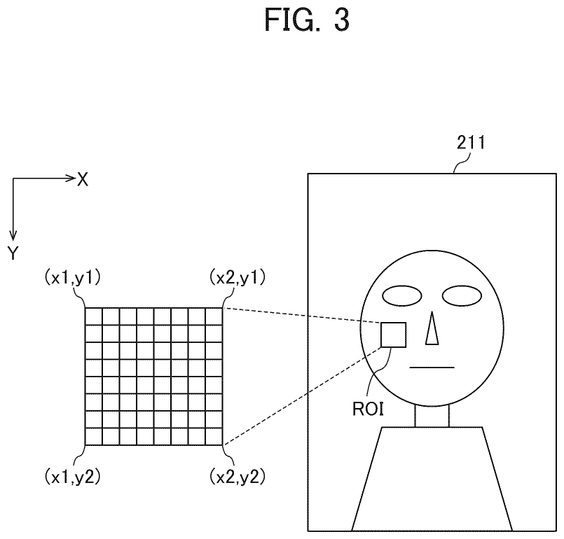

FIG. 3 illustrates one example of the region of interest ROI.

The measurement device 100 according to this embodiment calculates the biological information 212 from the pixel values of the pixels included in the region of interest ROI set in the image 211.

It is noted that to calculate the biological information 212, it is desirable to select and use, as the region of interest ROI, an image of a site suitable for calculation, such as a body surface site that is exposed to facilitate observing a pixel value change in a body surface image resulting from, for instance, a volume change in blood vessel. For instance, a cheek image of the living body 102 is suitable for calculating the biological information 212.

For example, the time-series signal obtaining unit 205 sets the region of interest ROI within the image 211 by using a face detection algorithm. To be specific, the time-series signal obtaining unit 205 extracts the amount of facial feature from the image 211 by using a face detection algorithm based on pattern recognition, machine learning, or other means. For instance, the amount of facial feature indicates the positions and shapes of the nose and eyes. The time-series signal obtaining unit 205 then sets the region of interest ROI on the basis of the extracted amount of facial feature. The time-series signal obtaining unit 205 can obtain the set position of the region of interest ROI as coordinates in the image 211; for example, the region of interest ROI illustrated in FIG. 3 is rectangular, and the coordinates of its vertices are denoted as (x1, y1), (x1, y2), (x2, y1), and (x2, y2) in the image 211.

The size of the face image of the living body 102 included in the image 211 may change in the case of a hand-induced shake of the living body 102 holding the measurement device 100, or a body motion of the living body 102. To be specific, the size of the face image included in the image 211 changes depending on the distance between the measurement device 100 and living body 102. For example, the further the living body 102 is away from the measurement device 100, the smaller the face image included in the image 211 becomes. As a result, the number of pixels within the region of interest ROI included in the image 211 decreases.

If there is an insufficient number of pixels within the region of interest ROI, the quality of the time-series biological signal 217 may degrade. That is, a pulse signal obtained at a time point when the number of pixels, which is hereinafter referred to as a pixel number P, within the region of interest ROI is equal to or smaller than a threshold Pth that is the determination threshold 215 may be unsuitable for accurately calculating the biological information 212. Accordingly, it is desirable that the biological-information calculating unit 206 do not use this pulse signal to calculate the biological information 212. Let the determination condition be so defined that the pixel number P within the region of interest ROI is greater than the threshold Pth, which is herein a value of 25. The pulse determining unit 207 in this case determines not to adopt a pulse signal including a biological signal value obtained at a time point when the pixel number P within the region of interest ROI is equal to or smaller than 25.

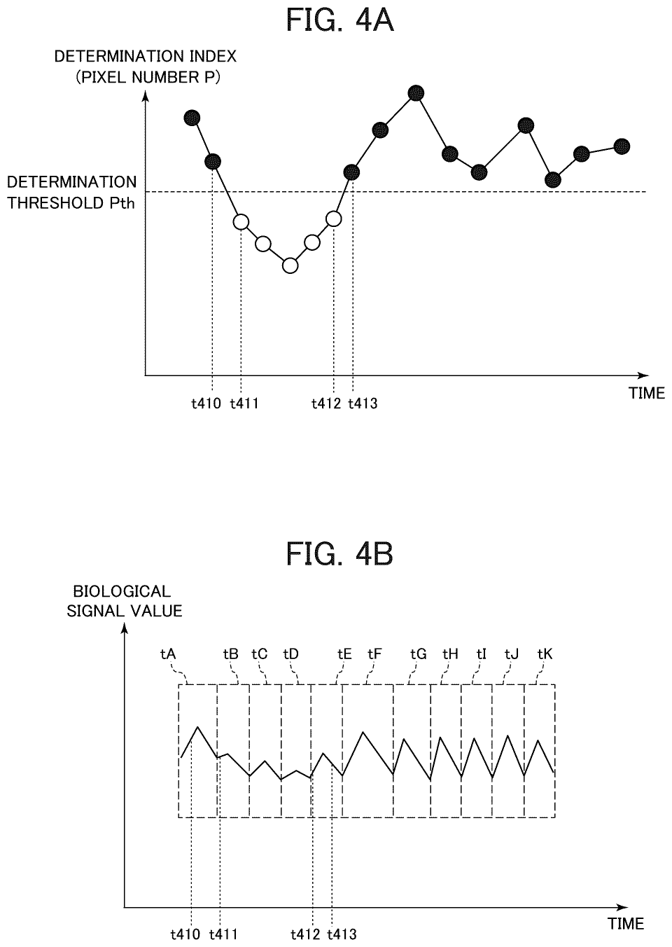

The process in the pulse determining unit 207 will be detailed with reference to FIGS. 4A and 4B. FIG. 4A is a graph illustrating, by way of example, transition of the determination index that indicates the pixel number P within the region of interest ROI, and the threshold Pth that is the determination threshold 215. In FIG. 4A, the lateral axis represents time, and the longitudinal axis represents the pixel number P, which is a determination index, within the region of interest ROI. Each point shown in the graph illustrated in FIG. 4A indicates the pixel number P within the region of interest ROI included in the image 211 obtained at mutually different time points.

FIG. 4B is a graph illustrating, by way of example, pulse signals at time points shown in the transition of the pixel number P within the region of interest ROI illustrated in FIG. 4A. In FIG. 4B, the lateral axis represents time, and the longitudinal axis represents biological signal value. For example, when the time-series biological signal 217 indicates a pulse wave signal, the biological signal value illustrated in FIG. 4B indicates pulse wave intensity. In FIG. 4B, 11 pulse signals are denoted by signs tA to tK, respectively, by way of example. The obtainment interval of the determination index shown in the longitudinal axis in FIG. 4A and the calculation interval of the biological signal value shown in the longitudinal axis in FIG. 4B are not limited to the foregoing as long as one or more determination indices are obtained for at least each pulse signal. For example, the biological signal value and determination index may be calculated for each frame of the image 211; alternatively, a plurality of determination indices may be obtained per pulse signal, and their average may be calculated. A disturbance, such as a body motion of the living body 102, can be observed in temporally more detail along with decrease in the obtainment time interval of the determination index. However, a shorter time interval than necessary complicates the calculation. It is hence desirable to determine the time interval in accordance with a desired calculation accuracy of the biological signal. It is noted that in this embodiment, both of the biological signal value and determination index are calculated for each frame of the image 211, and that the determination index and the biological signal temporally associated with the determination index are obtained at the same time point; in other words, the obtainment interval of the determination index and the calculation interval of the biological signal are identical. However, for the sake of description, the obtainment interval of the determination index illustrated in FIG. 4A is not identical to the calculation interval of the biological signal illustrated in FIG. 4B.

In this embodiment, as illustrated in FIG. 4B, the biological-information calculating unit 206 divides a signal that varies temporally based on the cycle of a biological phenomenon of the living body 102, at predetermined times based on the cycle. Some biological phenomena have periodic variation reflecting the conditions of a living body, such as respiration, heartbeat, blood pressure. For example, when the time-series biological signal 217 reflects information about the blood vessel of the living body 102, the intensity of the time-series biological signal 217 also fluctuates temporally in accordance with the cycle of blood vessel pulsation caused by the heart periodically repeating contraction and expansion, and has a cycle of repetition of increase and decrease. Since an actual biological phenomenon sensitively reacts to health or mental conditions, its cycle is not necessarily constant and is sometimes disturbed as in arrhythmia for example. However, since most biological phenomena vary with time in accordance with a certain degree of cycle, the biological-information calculating unit 206 can divide the time-series biological signal 217 at the predetermined times. It is noted that a biological signal obtained at a time point during which the biological signal is disturbed to such an extent that it cannot be divided may be regarded as being of poor quality and may be thus not used, and that the biological-information calculating unit 206 may divide, at the predetermined times, the time-series biological signal 217 other than the signal at that point.

In this way, the biological-information calculating unit 206 in this embodiment divides the time-series biological signal 217 into a plurality of pulse signals on the basis of the cycle of a biological phenomenon of the living body 102. To be specific, as denoted by the pulse signals tA to tK illustrated in FIG. 4B, the biological-information calculating unit 206 divides the time-series biological signal 217 into pulse signals at predetermined times based on a single cycle of fluctuation in the biological signal value. Let the pulse signals illustrated in FIG. 4B indicate pulse wave signals, and let the type of the biological information 212 be heartbeat or blood pressure. In this case, as illustrated in FIG. 4B, the biological-information calculating unit 206 sets, as a single cycle, a period from the rising edge to falling edge of the pulse wave accompanying the heart's contraction and expansion. FIG. 4B shows pulse signals divided in respective sections each partitioned by a rectangular dotted line, which denotes a signal cycle.

As described above, in order to measure the time-series biological signal 217 to calculate the biological information 212, the biological-information calculating unit 206 temporally divides the time-series biological signal 217, which varies with time, into a plurality of pulse signals on the basis of the cycle of a biological phenomenon of the living body 102. The biological-information calculating unit 206 then adopts pulse signals satisfying the determination condition from among these pulse signals and calculates the biological information 212 by using the adopted pulse signals. The biological-information calculating unit 206 can consequently calculate the biological information 212 accurately on the basis of the cycle of the biological phenomenon.

In the graphs illustrated in FIGS. 4A and 4B, the pixel number P that is a determination index at a time point before a time point t410 inclusive and after a time point t413 inclusive exceeds the threshold Pth. The pulse determining unit 207 accordingly adopts pulse signals associated with the time point before the time point t410 inclusive and after the time point t413 inclusive. To be specific, the pulse determining unit 207 adopts the pulse signals tA and tF to tK.

On the other hand, the pixel number P that is a determination index at a time point ranging from a time point t411 to a time point t412 both inclusive is equal to or smaller than the threshold Pth that is the determination threshold 215. The pulse determining unit 207 accordingly does not adopt pulse signals including biological signal values associated with the time point ranging from the time point t411 to the time point t412 both inclusive. To be specific, the pulse determining unit 207 does not adopt the pulse signals tB to tE. It is noted that the pulse signal tE partly includes a time point when the pixel number P is equal to or smaller than the threshold Pth. Hence, the pulse determining unit 207 does not adopt the pulse signal tE.

Then, information that enables identifying which of all the pulse signals are adopted pulse signals is stored as adopted-pulse information. The signal correcting unit 208 generates the correction signal 218 based on the adopted-pulse information. The signal correcting unit 208 can consequently generate the correction signal 218 formed from the adopted pulse signals.

The biological-information calculating unit 206 calculates the biological information 212 from the correction signal 218. To be specific, the biological-information calculating unit 206 calculates the biological information 212 by using a pulse signal temporally associated with a time point when the determination index exceeds the threshold Pth. That is, the biological-information calculating unit 206 calculates the biological information 212 from the image 211 by using the pulse signals tA and tF to tK illustrated in FIG. 4B.

It is noted that a lower threshold Pth may lead to the biological information 212 of lower quality. On the other hand, a higher threshold Pth leads to reduction in the number of pixels within the region of interest ROI resulting from a hand-induced shake of the living body 102 holding the measurement device 100 or a body motion of the living body 102; accordingly, the determination index does not satisfy the determination condition, and the pulse determining unit 207 is less likely to adopt a pulse signal. This may result in a long time that is required from when the imaging unit 101 starts imaging the living body 102 and to when the biological information 212 is output. It is hence preferable that the threshold Pth be set in accordance with pixel quality allowed for the biological information 212, and waiting time allowed by the living body 102.

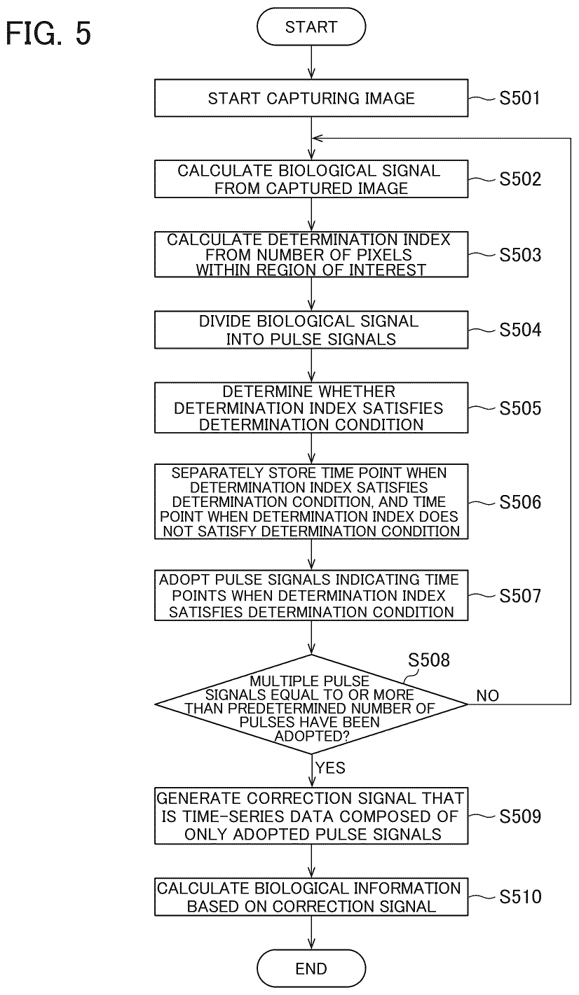

FIG. 5 is a flowchart showing one example of the operation of the measurement device 100 according to this embodiment.

In Step S501, the imaging unit 101 starts imaging the living body 102 to capture the image 211.

In Step S502, the time-series signal obtaining unit 205 calculates a biological signal from the image 211 captured in Step S501. To be specific, in Step S502, the time-series signal obtaining unit 205 obtains the time-series biological signal 217, which is temporal variation in a biological signal that can be calculated from the image 211 captured by the imaging unit 101. For instance, the time-series signal obtaining unit 205 sets the region of interest ROI within the image 211 and calculates the time-series biological signal 217 from the RGB pixel values of the pixels included in the region of interest ROI by using a calculation formula stored in the storage unit 203 in advance. Further, if necessary, the time-series signal obtaining unit 205 may obtain the time-series biological signal 217 indicating a pulse wave converted into, for instance, light absorbance, from temporal variation of a value calculated by substituting the luminance value of the pixels within the region of interest ROI of the image 211 into a predetermined mathematical formula. Alternatively, the time-series signal obtaining unit 205 may obtain the time-series biological signal 217 indicating a pulse wave, through independent component analysis, biogenic component separation, or other methods.

In Step S503, the time-series signal obtaining unit 205 calculates a determination index indicating the pixel number P within the region of interest ROI, to obtain the determination index signal 216, which is time-series data indicating temporal variation in the determination index calculated from the image 211.

In Step S504, the biological-information calculating unit 206 divides the time-series biological signal 217 into pulse signals. To be specific, the biological-information calculating unit 206 divides the time-series biological signal 217 into a plurality of pulse signals at predetermined times based on a single cycle of a biological phenomenon of the living body 102. The cycle based on the biological phenomenon of the living body 102 for pulse signal division is set in accordance with the type of the biological information 212 to be measured.

In Step S505, the pulse determining unit 207 determines whether the determination index satisfies a determination condition. The determination condition is that the number of pixels within the region of interest ROI indicated by the determination index exceeds the determination threshold 215.

That is, the pulse determining unit 207 determines whether the pixel number P is greater than the threshold Pth. If the pixel number P within the region of interest ROI exceeds the threshold Pth, the pulse determining unit 207 determines that the determination index satisfies the determination condition. On the other hand, if the pixel number P within the region of interest ROI is equal to or smaller than the threshold Pth, the pulse determining unit 207 determines that the determination index does not satisfy the determination condition. The pulse determining unit 207 determines whether the determination index satisfies the determination condition, for each time point of the determination index signal 216, which is time-series data.

It is noted that when the storage unit 203 stores a plurality of determination thresholds 215, the pulse determining unit 207 may select one of the determination thresholds 215 in measurement. It is also noted that the pulse determining unit 207 may change the determination threshold 215 that is to be used, upon the input unit 201 receiving an operation to change the determination threshold 215 that is to be used among the plurality of determination thresholds 215 stored in the biological-information storing unit 214. For example, the pulse determining unit 207 may use an optimal determination threshold for each user among determination thresholds that are set in accordance with a plurality of users, and that are stored in the biological-information storing unit 214, so that the biological information 212 can be obtained more accurately.

In Step S506, the control unit 204 stores the time point when the determination index satisfies the determination condition, and the time point when the determination index does not satisfy the determination condition separately in the measurement-information storing unit 213 so that they can be distinguished from each other. Examples of information indicating the time points include time, and identification information associated with the time points. It is noted that the measurement-information storing unit 213 may not necessarily store the time point when the determination index does not satisfy the determination condition.

In Step S507, the pulse determining unit 207 adopts pulse signals associated with the time points when the determination index satisfies the determination condition. To be specific, the pulse determining unit 207 adopts pulse signals with their biological signal values calculated from the pixel values within the region of interest ROI of an image captured at the time points when the determination index satisfies the determination condition, and the pulse determining unit 207 stores adopted-pulse information relating to the adopted pulse signals.

The accuracy of the biological information 212 calculated by the biological-information calculating unit 206 may not be guaranteed when the imaging unit 101 does not image the living body 212 for a predetermined time required for appropriately calculating the biological information 212. Likewise, the accuracy of the biological information 212 calculated by the biological-information calculating unit 206 may not be guaranteed when the total number of adopted pulse signals is less than a predetermined number of pulses required for appropriately calculating the biological information 212.

Accordingly, in Step S508, the pulse determining unit 207 determines whether pulse signals equal to or more than the predetermined number of pulses have been adopted. To be specific, the pulse determining unit 207 determines whether the living body 102 has been imaged, and whether pulse signals equal to or more than the predetermined number of pulses have been adopted. The predetermined number of pulses is stored in the measurement-information storing unit 213 in advance. The predetermined number of pulses may be different depending on the type of the biological information 212 to be measured. Alternatively in Step S508, the pulse determining unit 207 may determine whether the number of pulse signals whose total time satisfies a predetermined time or longer have been adopted. In this case, the predetermined time is stored in the measurement-information storing unit 213 in advance. That is, in Step S508, the pulse determining unit 207 adopts a plurality of pulse signals satisfying at least one of the predetermined number of pulses or more, and the predetermined time or longer.

If pulse signals satisfying at least one of the predetermined number of pulses or more, and the predetermined time or longer are not adopted in Step S508, the control unit 204 returns the process to Step S502. In the process after Step S502, the pulse determining unit 207 adds, to an already adopted pulse signals, pulse signals adopted anew on or after the time point associated with the already adopted pulse signal, and the pulse determining unit 207 stores the adopted-pulse information in the measurement-information storing unit 213. This enables the measurement device 100 to reduce a time required for adopting the number of pulse signals satisfying at least one of the predetermined number of pulses or more, and the predetermined time or longer.

On the other hand, if the number of pulse signals satisfying at least one of the predetermined number of pulses or more, and the predetermined time or longer is adopted in Step S508, the signal correcting unit 208 in Step S509 generates the correction signal 218 or time-series data composed of only the adopted pulse signals. Accordingly, the signal correcting unit 208 generates the correction signal 218 including pulse signals with their biological signal values associated with the time points when the determination index satisfies the determination condition.

In Step S510, the biological-information calculating unit 206 calculates the biological information 212 from the correction signal 218. That is, the biological-information calculating unit 206 calculates the biological information 212 by using the pulse signals adopted by the pulse determining unit 207. To be specific, the biological-information calculating unit 206 calculates the biological information 212 by using the pulse signals adopted by the pulse determining unit 207 as satisfying at least one of the predetermined number of pulses or more, and the predetermined time or longer. Then, the output unit 202 outputs the biological information 212, as necessary. For example, the output unit 202 outputs the biological information 212 so as to display the biological information 212 on a display device (not shown) connected to the measurement device 100. Then, the control unit 204 ends the process.

As described above, the measurement device 100 according to this embodiment determines whether each pulse signal included in the time-series biological signal 217 is appropriate. To be specific, the measurement device 100 does not adopt a pulse signal associated with a time point when the pixel number P within the region of interest ROI decreases so significantly that the determination index no longer exceeds the determination threshold 215, thus not satisfying the determination condition, but adopts a pulse signal of high quality associated with a time point when the pixel number P exceeds the determination threshold 215.

Calculating the biological information 212 by the use of biological signal values included in pulse signals of high quality can improve the calculation accuracy further than calculating the biological information 212 by the use of all the biological signal values included in the time-series biological signal 217.

At this time, performing a determination for each pulse signal enables the biological information 212 to be calculated by the use of a biological signal whose values based on a single cycle of a biological phenomenon are complete, and that is thus suitable for measuring the biological information 212. As a result, the measurement device 100 according to this embodiment can accurately calculate the biological information 212 reflecting the cycle of the biological phenomenon of the living body 102.

It is noted that the pulse determining unit 207 may determine whether the time point when the determination index satisfies the determination condition continues for a certain time or longer. If the time point when the determination index satisfies the determination condition continues for the certain time or longer, the pulse determining unit 207 may then adopt a pulse signal indicating a biological signal value calculated from the image 211 captured at the time point when the determination index satisfies the determination condition. As a result, the measurement device 100 according to this embodiment can accurately calculate the biological information 212 by the use of a biological signal temporally continuously reflecting the cycle of a biological phenomenon of the living body 102.

Further, among information pieces to be stored in the storage unit 203, information other than information pieces that need to be stored, such as a user log, does not necessarily have to be stored over the long term; for example, the time when the determination index satisfies the determination condition, or identification information about the adopted pulse signals may be stored in such a manner as to be stored in the form of a variable temporarily during the calculation of the biological information 212, and it may be deleted after the calculation is completed. Alternatively, such information may remain stored even after the calculation is completed; information stored over the long term can be used in, but not limited to, data analysis for enhancing the calculation accuracy of the biological information 212.

### First Modification

The first modification of the first embodiment will be described. It is noted that identical or like constituents will be denoted by the same signs throughout the drawings, and the descriptions of redundancies will be omitted.

The position of the region of interest ROI within the image 211 is shifted significantly in some cases at, for instance, a time point of occurrence of a hand-induced shake of the living body 102 holding the measurement device 100, or a time point of occurrence of a body motion of the living body 102. To be more specific, the coordinates of the region of interest ROI within the image 211 vary significantly in some cases. It is noted that the coordinates here are a position within the image 211. Such a variation caused by a hand-induced shake, a body motion, or other things becomes a disturbance, which can cause a biological signal value and a pulse signal value to vary. Accordingly, the time-series signal obtaining unit 205 according to this modification calculates a determination index from a displacement of the coordinates of the region of interest ROI within the image 211 captured at different time points. For instance, the time-series signal obtaining unit 205 according to this modification calculates a determination index from a displacement between different frames at each of some or all of coordinates (x1, y1), (x1, y2), (x2, y1), and (x2, y2), which are the coordinates of the vertices of the region of interest ROI illustrated in FIG. 3. That is, the determination index according to this modification indicates a displacement of the coordinates of a region of interest set within the image 211 captured at different time points. A determination condition according to this modification is that the coordinate displacement is equal to or smaller than the determination threshold 215 (a second determination threshold) according to this modification.

Let the coordinates of the vertex at the upper left corner of the region of interest ROI included in the image 211 in an i-th frame be denoted as (x1i, y1i). Here, i is a natural number equal to or greater than 1. In addition, let the coordinates of the vertex at the upper left corner of the region of interest ROI included in the image 211 in an n-th frame be denoted as (x1n, y1n). Here, n is a natural number equal to or greater than i+1. The displacement of the vertex at the upper left corner of the region of interest ROI between the i-th and n-th frames is denoted as (x1n−x1i, y1n−y1i). Furthermore, let the determination threshold 215 according to this modification indicate xth, which is a threshold for the x-coordinates in the region of interest ROI, and yth, which is a threshold for the y-coordinates in the region of interest ROI. In x1n−x1i≤xth and y1n−y1i≤yth, the pulse determining unit 207 determines that the determination index satisfies the determination condition, and adopts pulse signals associated with time points when the i-th to n-th frames are captured. On the other hand, in x1n−x1i>xth and y1n−y1i>yth, the pulse determining unit 207 determines that the determination index does not satisfy the determination condition, and does not adopt the pulse signals associated with the time points when the i-th to n-th frames are captured.

As described above, the measurement device 100 according to this modification does not use a biological signal value obtained at a time point when the coordinates of the region of interest ROI vary beyond the determination threshold 215, for calculating the biological information 212. As a result, the measurement device 100 according to this modification can accurately calculate the biological information 212 reflecting the cycle of a biological phenomenon of the living body 102 even in the case of occurrence of a hand-induced shake or a body motion of the living body 102 holding the measurement device 100 while the imaging unit 101 is imaging the living body 102.

It is noted that the foregoing determination condition according to this modification is a mere example and is not restrictive. For example, the determination condition may be x1n−x1i≤xth or y1n−y1i≤yth. Further, the determination condition may be indicated only by displacements in the x-coordinates in the region of interest ROI. Further, when the region of interest ROI is rectangular, the determination condition may be indicated by displacements in the coordinates of its four corners. Further, the determination condition may be set in accordance with the type of the biological information 212 to be measured, or an accuracy required for the biological information 212. This modification has described, by way of example, that a displacement of the region of interest ROI occurring when it moves is expressed using the coordinates of the region of interest ROI in the image 211. Any index indicating a movement of the region of interest ROI may be used; a desirable index is one expressing a displacement as a numeric value, such as coordinates.

### Second Modification

The second modification of the first embodiment will be described. It is noted that identical or like constituents will be denoted by the same signs throughout the drawings, and the descriptions of redundancies will be omitted.

In response to a motion of the living body 102, such as eye blinking, the face skin of the living body 102 moves, and a sudden fluctuation in biological signal value may occur like spike noise. Such a disturbance causes the quality of the time-series biological signal 217 obtained during the eye blinking of the living body 102 to become lower than the quality of the time-series biological signal 217 obtained at a time point during which the living body 102 is not producing motions, such as eye blinking. Accordingly, the time-series signal obtaining unit 205 according to this modification calculates a determination index indicating the number of motions of the living body 102 per unit time calculated from the image 211. For example, the number of motions indicated by the determination index is the number of eye blinks per unit time.

To be specific, the time-series signal obtaining unit 205 according to this modification calculates, for each pulse signal, the number of motions of the living body 102 per unit time within a time period during which biological signals included in a single pulse signal are obtained. For example, the time-series signal obtaining unit 205 according to this modification calculates, as the determination index, the number of eye blinks per unit time in an image of the eyelids included in the image 211. A determination condition according to this modification is that the number of motions of the living body 102 per unit time is equal to or smaller than the determination threshold 215 (a third determination threshold) according to this modification. For instance, the determination condition according to this modification is that the number of eye blinks of the living body 102 per unit time is equal to or smaller than the determination threshold 215.

The determination threshold 215 according to this modification indicates a threshold for the number of motions per unit time. For instance, the determination threshold 215 according to this modification indicates a threshold for the number of eye blinks per unit time. In this case, if the number of eye blinks indicated by the determination index exceeds the threshold for the number of eye blinks indicated by the determination threshold 215, the pulse determining unit 207 according to this modification determines that the determination index does not satisfy the determination condition. The pulse determining unit 207 does not thus adopt pulse signals calculated from the image 211 obtained at time points included in a time period during which the number of eye blinks exceeds the threshold for the number of eye blinks indicated by the determination threshold 215.

As described above, upon the number of motions of the living body 102, such as eye blinks, exceeding the determination threshold 215, the measurement device 100 according to this modification does not use a biological signal value calculated from an image captured during the time period of the motions, for calculating the biological information 212. As a result, the measurement device 100 according to this modification can accurately calculate the biological information 212 reflecting the cycle of a biological phenomenon of the living body 102, by excluding biological signal values obtained during a time period of motions, such as eye blinks, even when the living body 102 produces motions, such as eye blinks, a number of counts exceeding the determination threshold 215 per unit time.

### Second Embodiment

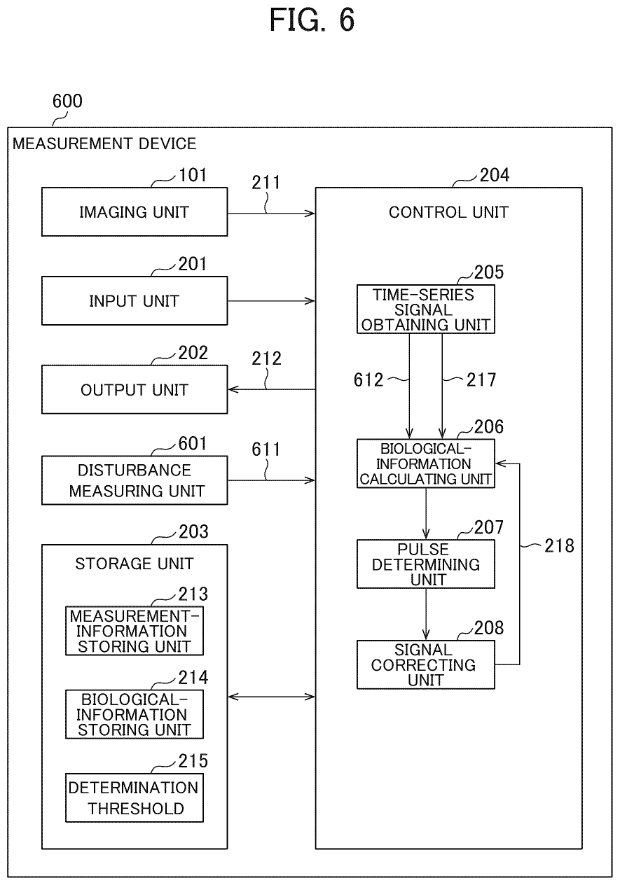

The second embodiment will be described with reference to FIGS. 6 to 8. It is noted that identical or equivalent constituents will be denoted by the same signs throughout the drawings, and the descriptions of redundancies will be omitted.

FIG. 6 is a block diagram illustrating one example of the configuration of a measurement device 600 according to this embodiment. The measurement device 600 illustrated in FIG. 6 is different from the measurement device 100 illustrated in FIG. 2 in that the measurement device 600 includes a disturbance measuring unit 601 illustrated in FIG. 6.

When the imaging unit 101 images the living body 102 producing a body motion with the measurement device 600 held by the living body 102, the measurement device 600 moves due to the body motion of the living body 102. A body motion of the living body 102 holding the measurement device 600, such as a hand-induced shake, a sneeze, a cough, or swaying, becomes a disturbance, so that a noise or an undesirable variation may be mixed to a biological signal calculated from the image 211.

Accordingly, the measurement device 600 includes the disturbance measuring unit 601 that obtains disturbance information 611 indicating the amount of variation in the measurement device 600 varying spatially based on, but not limited to, a body motion of the living body 102 or the intensity of the body motion. The disturbance information 611 indicates at least one selected from the group consisting of a displacement of the measurement device 600 per unit time, the amount of velocity variation in the measurement device 600, the amount of acceleration variation in the measurement device 600, and the amount of angular-velocity variation in the measurement device 600. The measurement device 600 includes a device that can measure the amount of spatial variation in the measurement device 600 as time-series data, and the measurement device 600 obtains the disturbance information 611, which is time-series data of the amount of spatial variation measured by this device. Examples of the device that can measure the amount of spatial variation in the measurement device 600 include an accelerometer and a gyroscope, both of which can measure position, acceleration, angular velocity, and other things.

The time-series signal obtaining unit 205 according to this embodiment calculates a determination index obtained from the disturbance information 611 and obtains a determination index signal 612, which is time-series data of the determination index. That is, the determination index according to this embodiment indicates the amount of spatial variation in the measurement device 600 and is temporally associated with a pulse signal including biological signal values obtained from the image 211. A determination condition according to this embodiment is that the amount of spatial variation in the measurement device 600 is equal to or smaller than the determination threshold 215 (a fourth determination threshold) according to this embodiment.

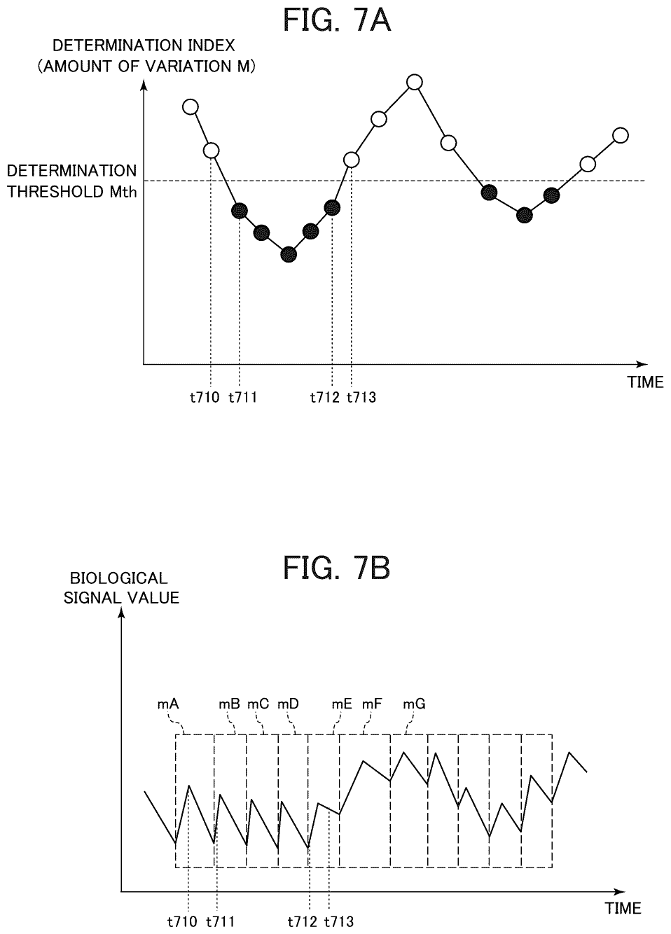

The process in the pulse determining unit 207 according to this embodiment will be detailed with reference to FIGS. 7A and 7B.

FIG. 7A is a graph illustrating, by way of example, transition of the determination index indicating an amount of variation M in the measurement device 600, and a threshold Mth being the determination threshold 215. In FIG. 7A, the lateral axis represents time, and the longitudinal axis represents the amount of variation M, which is indicated by the determination index. The points shown in the graph illustrated in FIG. 7A each indicate the amount of variation M indicated by the disturbance information 611 obtained at mutually different time points.

FIG. 7B is a graph illustrating, by way of example, pulse signals at time points shown in the transition of the amount of variation M illustrated in FIG. 7A. In FIG. 4B, the lateral axis represents time, and the longitudinal axis represents biological signal value. It is noted that in FIG. 7B, although the measurement still continues after a pulse signal mG, it will be omitted for description. FIG. 7B, which is similar to FIG. 4B other than the foregoing, will not be detailed.

In pulse signals mA to mG in the graphs illustrated in FIGS. 7A and 7B, since the amount of variation M indicated by the disturbance information 611 at a time point before the time point t710 inclusive and after the time point t713 inclusive is larger than the threshold Mth, which is the determination threshold value 215, the pulse determining unit 207 does not adopt pulse signals including biological signal values calculated from the image 211 captured at the time point before the time point t710 inclusive and after the time point t713 inclusive. To be specific, the pulse determining unit 207 determines not to adopt the pulse signal mA and the pulse signals mE to mG among the pulse signals mA to mG.

On the other hand, since the amount of variation M indicated by the determination index at a time point ranging from a time point t711 to a time point t712 both inclusive is equal to or smaller than the threshold Mth, which is the determination threshold value 215, the pulse determining unit 207 adopts pulse signals including biological signal values calculated from the image 211 captured at the time point ranging from the time points t711 to t712 both inclusive. To be specific, the pulse determining unit 207 adopts the pulse signals mB to mD. It is noted that the pulse determining unit 207 determines to adopt pulse signals including only biological signal values calculated from an image captured at time points when the amount of variation M, which is indicated by the disturbance information 611, is equal to or smaller than the threshold Mth. For instance, the pulse determining unit 207 does not adopt the pulse signal mE because the pulse signal mE in FIG. 7B, which includes both of the time point t712 when the amount of variation M, indicated by the disturbance information 611, is equal to or smaller than the threshold Mth, and the time point t713 when the amount of variation M is equal to or greater than the threshold Mth, includes at least one time point when the amount of variation M is equal to or greater than the threshold Mth.

Accordingly, the biological-information calculating unit 206 calculates the biological information 212 from the image 211 by using the pulse signals mB to mD illustrated in FIG. 7B. That is, the biological-information calculating unit 206 calculates the biological information 212 by using pulse signals including biological signal values calculated from an image at time points when the determination index is equal to or smaller than the threshold Mth.

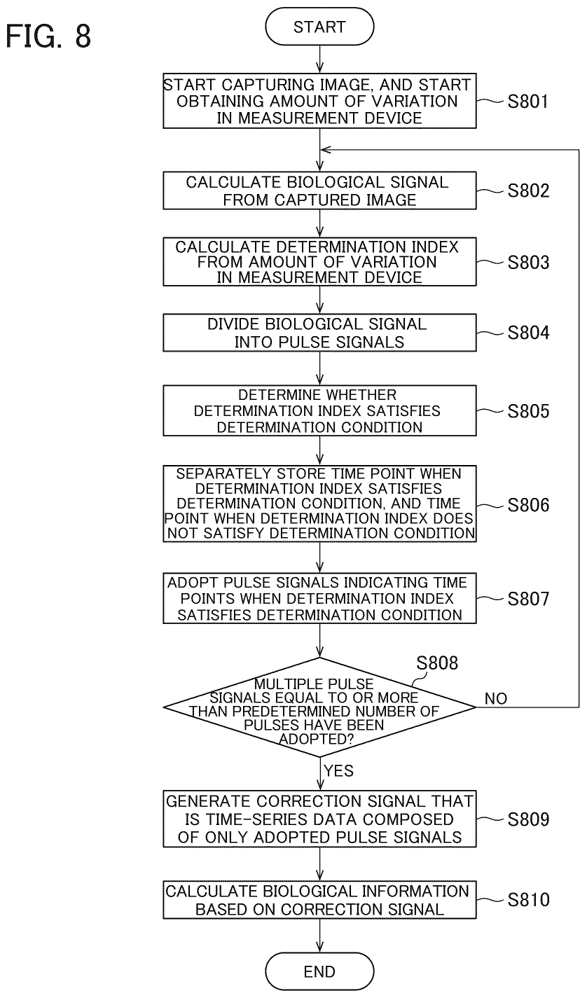

FIG. 8 is a flowchart showing one example of the operation of the measurement device 600 according to this embodiment. It is noted that process steps similar to those shown in FIG. 5 will not be detailed.

The difference between the flowchart shown in FIG. 8 and the flowchart shown in FIG. 5 lies in that the amount of spatial variation in the measurement device 600, which is indicated by the disturbance information 611 obtained by the disturbance measuring unit 601, is used as a determination index.

In Step S801, the imaging unit 101 starts capturing the image 211, and the disturbance measuring unit 601 starts obtaining the amount of variation in the measurement device.

In Step S802, the time-series signal obtaining unit 205 obtains the time-series biological signal 217 from the image 211 captured by the imaging unit 101.

In Step S803, the time-series signal obtaining unit 205 calculates a determination index from the amount of variation indicated by the disturbance information 611, to obtain the determination index signal 216 as its time-series data.

In Step S804, the biological-information calculating unit 206 divides the time-series biological signal 217 into pulse signals. Step S804, which is similar to Step S504 shown in FIG. 5, will not be detailed.

In Step S805, the pulse determining unit 207 determines whether the determination index calculated in Step S803 satisfies a determination condition. The determination condition according to this embodiment is that the amount of variation M, which is indicated by the disturbance information 611, is equal to or smaller than the threshold Mth. That is, the pulse determining unit 207 determines whether the amount of variation M, which is indicated by the disturbance information 611, is equal to or smaller than the threshold Mth. If the amount of variation M, which is indicated by the disturbance information 611, is equal to or smaller than the threshold Mth, the pulse determining unit 207 determines that the determination index satisfies the determination condition. On the other hand, if the amount of variation M, which is indicated by the disturbance information 611, is greater than the threshold Mth, the pulse determining unit 207 determines that the determination index does not satisfy the determination condition. The pulse determining unit 207 determines whether the determination index satisfies the determination condition, for each time point of the determination index signal 216, which is time-series data. Then, the control unit 204 moves the process to Step S806. Step S806 and the subsequent process, which are similar to Step S506 and the subsequent process shown in FIG. 5, will not be detailed.

It is noted that the foregoing is one aspect; the pulse determining unit 207 may use disturbance information obtained from an image captured by the imaging unit 101, to determine whether this disturbance is really undesirable. That is, the pulse determining unit 207 may determine, as an undesirable disturbance, a disturbance obtained from an image at the almost same timing as a disturbance detected by the disturbance measuring unit 601. To be specific, when the time difference between a time point of a large body motion, such as a large facial motion of the living body 102 that can be detected from an image included in the image 211, and a time point when the amount of variation M, which is indicated by the disturbance information 611, does not satisfy the determination condition is smaller than a predetermined range, the pulse determining unit 207 does not adopt pulse signals including these time points. In this case, comparing the information obtained from the image with the information obtained from the disturbance measuring unit 601 can determine whether the disturbance is really undesirable, thereby enabling the effect of the disturbance to be eliminated more accurately.

As described above, the measurement device 600 according to this embodiment can accurately calculate the biological information 212 reflecting the cycle of a biological phenomenon of the living body 102 by more accurately eliminating the effect of a body motion of the living body 102.

The individual processes executed in the foregoing embodiments are not limited to the process aspects exemplified in the respective embodiments. The foregoing functional blocks may be implemented by using either a logic circuit (hardware) formed in an integrated circuit or other things, or software using a CPU. The individual processes executed in the foregoing embodiments may be executed by a plurality of computers. For instance, the process executed by the control unit 204 may be partly executed by another computer, or the whole process may be shared by a plurality of computers.

The present disclosure is not limited to the above-described embodiments. The present disclosure may be replaced with a configuration substantially identical to that described in the above-described embodiments, a configuration that provides the same action and effect, or a configuration that can achieve the same object. In the present disclosure, an embodiment that is obtained in combination, as appropriate, with the technical means disclosed in the respective embodiments is also encompassed within the technical scope of the present disclosure. Furthermore, combining the technical means disclosed in the respective embodiments can form a new technical feature.

While there have been described what are at present considered to be certain embodiments of the disclosure, it will be understood that various modifications may be made thereto, and it is intended that the appended claim cover all such modifications as fall within the true spirit and scope of the disclosure.

What is claimed is:

1. A measurement device comprising:
an imaging unit configured to image a living body to capture an image as time-series data;
a time-series signal obtaining unit configured to obtain a time-series biological signal indicating a temporal variation in a biological signal, and obtain a determination index temporally associated with the biological signal, the biological signal being a value relating to the living body and calculated from the image;
a biological-information calculating unit configured to extract a pulse signal by dividing the time-series biological signal at a predetermined time based on a cycle of a biological phenomenon of the living body; and
a pulse determining unit configured to determine whether the determination index satisfies a predetermined determination condition, and adopt the pulse signal temporally associated with the determination index satisfying the predetermined determination condition,
wherein the biological-information calculating unit calculates biological information relating to the living body by using the pulse signal adopted by the pulse determining unit.

2. The measurement device according to claim 1, wherein at time points each indicating a time of capturing of the image with a value of the biological signal calculated, the value of the biological signal is calculated from a pixel value of a pixel within a region of interest included in the image captured at each of the time points.

3. The measurement device according to claim 2, wherein
the determination index indicates a number of pixels within the region of interest, and
the predetermined determination condition is that the number of pixels exceeds a first determination threshold.

4. The measurement device according to claim 1, wherein
the determination index indicates a displacement between the regions of interest included in the image captured at the time points different from each other, and
the predetermined determination condition is that the displacement is equal to or smaller than a second determination threshold.

5. The measurement device according to claim 1, wherein
the determination index indicates a number of motions of the living body per unit time calculated from the image, and
the predetermined determination condition is that the number of motions is equal to or smaller than a third determination threshold.

6. The measurement device according to claim 5, wherein the number of motions is a number of eye blinks per unit time.

7. The measurement device according to claim 1, further comprising a disturbance measuring unit configured to obtain disturbance information indicating an amount of spatial variation in the measurement device,
wherein the determination index indicates the amount of spatial variation, and
the predetermined determination condition is that the amount of spatial variation is equal to or smaller than a fourth determination threshold.

8. The measurement device according to claim 1, wherein the biological-information calculating unit calculates the biological information based on a plurality of pulse signals satisfying at least one of a predetermined number of pluses or more, and a predetermined time or longer.

9. A measurement method comprising:
imaging a living body to capture an image as time-series data;
obtaining a time-series biological signal indicating a temporal variation in a biological signal, and obtaining a determination index temporally associated with the biological signal, the biological signal being a value relating to the living body and calculated from the image;
extracting a pulse signal by dividing the time-series biological signal at a predetermined time based on a cycle of a biological phenomenon of the living body;
determining whether the determination index satisfies a predetermined determination condition; and
calculating biological information relating to the living body by using the pulse signal temporally associated with the determination index satisfying the predetermined determination condition.

10. The measurement device according to claim 2, wherein
the determination index indicates a displacement between the regions of interest included in the image obtained at the time points different from each other, and
the predetermined determination condition is that the displacement is equal to or smaller than a second determination threshold.

11. The measurement device according to claim 2, wherein
the determination index indicates a number of motions of the living body per unit time calculated from the image, and
the predetermined determination condition is that the number of motions is equal to or smaller than a third determination threshold.

12. The measurement device according to claim 11, wherein the number of motions is a number of eye blinks per unit time.

13. The measurement device according to claim 2, further comprising a disturbance measuring unit configured to obtain disturbance information indicating an amount of spatial variation in the measurement device,
wherein the determination index indicates the amount of spatial variation, and
the predetermined determination condition is that the amount of spatial variation is equal to or smaller than a fourth determination threshold.

14. The measurement device according to claim 2, wherein the biological-information calculating unit calculates the biological information based on a plurality of pulse signals satisfying at least one of a predetermined number of pluses or more, and a predetermined time or longer.
